(19) United States
(12) Patent Application Publication
(10) Pub. No.: US 2022/0378347 A1
(43) Pub. Date: Dec. 1, 2022

(54) PHANTOM FOR URODYNAMIC MEASUREMENT

(71) Applicant: SapienMed Co., Ltd, Seoul (KR)

(72) Inventors: Seung June OH, Seoul (KR); Ji Hoon PARK, Seoul (KR)

(73) Assignee: SapienMed Co., Ltd, Seoul (KR)

(21) Appl. No.: 17/571,507

(22) Filed: Jan. 9, 2022

Related U.S. Application Data
(60) Provisional application No. 63/193,609, filed on May 27, 2021.

(51) Int. Cl. A61B 5/20; A61B 5/00

(52) U.S. Cl. CPC A61B 5/205; A61B 5/6852; A61B 5/6874; A61B 2562/0247

(57) ABSTRACT

The present disclosure provides a urodynamic measurement apparatus that includes a urethral catheter that may be inserted into a patient's urinary bladder; a first sensor that is connected with the urethral catheter to measure an internal pressure of the urinary bladder; an abdominal catheter that may be inserted into a patient's rectum; a second sensor that is connected to the abdominal catheter to measure an abdominal pressure; and a first position adjusting part for adjusting heights of the first sensor and the second sensor according to the position of the patient's pubis.

### 1. FIELD

The present disclosure relates to a phantom for urodynamic measurement, and more specifically, to a phantom for urodynamic measurement, that may be used in educating medical staff in more detail about the urodynamic measurement and that may be used in a performance test of a urodynamic measurement apparatus.

### 2. BACKGROUND

Adequate storing and emptying of urine is achieved through integration of functions of the urinary bladder and urethral sphincter, and the nervous system that controls these organs.

Urodynamic measurement (or urodynamic study) is an in-depth examination for diagnosing functional abnormalities of the urinary bladder and urethral sphincter. Urodynamic measurement is a collective term for tests made up of several detailed items such as uroflowmetry, filling cystometry, pressure-flow study, urethral pressure profile, and urethral sphincter electromyography. These tests are conducted selectively for patients with voiding dysfunction and urine incontinence.

Medical staff can gain detailed information necessary to accurately identify the characteristics of lower urinary tract dysfunction through urodynamic measurement. Based on the results of urodynamic measurement, medical staff can clearly distinguish between various types of urinary bladder and urethral dysfunctions that can be expressed clinically even in a single disease entity.

The most important role of urodynamic measurement is to provide detailed information necessary for the medical staff to determine the prognosis of patients with specific urinary tract dysfunctions and establish a treatment plan. Based on the findings of the lower urinary tract dysfunction from the urodynamic measurement, medical staff can provide the best treatment method applicable to a patient.

Urodynamic measurement is the single most important test in determining the prognosis and treatment of the lower urinary tract dysfunction. Therefore, it is necessary for the medical staff to accurately interpret the results of a patient's urodynamic measurement.

FIG. 1 is a graph showing the intravesical pressure (Pves), abdominal pressure (Pabd), detrusor pressure (Pdet) and urethral sphincter electromyography (EMG) activity, measured during the filling cystometry of a patient in a supine position.

Referring to FIG. 1, urodynamic measurement includes several detailed measurements that identify various indicators related to the function of storing and emptying urine. Urodynamic measurement allows the medical staff to interpret the results of the intravesical pressure (Pves) 1, abdominal pressure (Pabd) 2, detrusor pressure (Pdet) 3, and urethral sphincter activity 4 information of the patient, measured through a urodynamic measurement apparatus as aforementioned, making it possible to diagnose the patient's lower urinary tract dysfunction.

Here, the detrusor pressure (Pdet) 3 cannot be measured directly by a urodynamic measurement apparatus, and thus it is displayed as a virtual value obtained by subtracting the abdominal pressure (Pabd) 2 from the intravesical pressure (Pves) 1 in real time through computer calculation.

In urodynamic measurement, securing and maintaining the quality of measurement is much more important compared to other general tests. That is because, in the process of urodynamic measurement, various kinds of unexpected errors and artifacts may occur more frequently than other tests, and in many cases, the occurrence of errors and artifacts cannot be avoided even if we do our best. Even in a normal measurement process, errors and artifacts caused by psychological factors such as the subject person's tension, errors and artifacts made by the examiner such as in initial setting, and errors and artifacts due to equipment factors and the like occur very frequently and in various ways.

Therefore, it is necessary to filter and correct these errors and artifacts, and obtain accurate test results. In order to minimize these test errors and artifacts, the International Continence Society (ICS) provides the Standardization and Good Urodynamic Practice as strict guidelines for testing and reading urodynamic measurements.

In most hospitals, dedicated manpower is assigned to urodynamic measurement, and depending on the examiner's work skill level or experience, there can be a big difference in the ability to identify and cope with the errors and artifacts during the measurement, in other words, there can be a big difference in the quality of the measurement. There are many cases where the final treatment method for the patient changes due to a change in the interpretation of the measurement result according to the experience and knowledge level of the medical staff in charge. Therefore, a lot of hands-on practical education on the practice of urodynamic measurement, such as insertion of urethral catheter and rectal catheter into the urethra and anus, sphincter electromyography, urethral pressure test, and interpretation of graphs, are required. However, in the practical education, introducing the catheter to the urethra and anus, which are sensitive body parts, should be sufficiently considered. It is a very difficult problem in reality to proceed with such practice and education through urodynamic measurement on patients. However, if a human simulation phantom model is used, effective hands-on workshop-type practice may be possible that not only reproduces the normal measurement process but also improves the ability to identify errors and artifacts and to cope with them. If the practical experience of medical staff is improved by such practical education through phantom, the quality of urodynamic measurement will also improve.

### SUMMARY

Therefore, a purpose of the present disclosure is to resolve the aforementioned problems of prior art, that is, to provide a phantom for urodynamic measurement, that can be utilized in the process of developing a urodynamic measurement apparatus, and in practices and education on urodynamic measurement for medical staff.

A phantom for urodynamic measurement that sends measurement stimulus to a urodynamic measurement apparatus, includes a lower urinary tract part for providing pressure to a urethral catheter of the urodynamic measurement apparatus; and a rectal part that is positioned adjacent to the lower urinary tract part, and that provides pressure to a rectal catheter of the urodynamic measurement apparatus.

The lower urinary tract part may include a urinary bladder bag; a first air discharge pipe connected to one end of the urinary bladder bag; a urethral hose connected to the other end of the urinary bladder bag; and a first sphincter part surrounding the urethral hose.

The phantom for urodynamic measurement may further include a case for accommodating the lower urinary tract part and the rectal part, and the case may include an auxiliary side surface cover for physically separating the lower urinary tract part and the rectal part from each other.

The rectal part may include a rectal bag; a second air discharge pipe connected to one end of the rectal bag; a guide hose connected to the other end of the rectal bag; and a second sphincter part that surrounds the guide hose.

The phantom for urodynamic measurement may include an electrical signal generator for generating an electrical signal to an electromyography electrode of the urodynamic measurement apparatus.

The phantom for urodynamic measurement may further include a posture adjusting part for adjusting an angle of the case and a height of the case.

The phantom for urodynamic measurement of the present disclosure may be used in education on urodynamic measurement for medical staff and in performance tests of a urodynamic measurement apparatus.

### BRIEF DESCRIPTION OF THE DRAWINGS

FIG. 1 is a graph showing the intravesical pressure (Pves), abdominal pressure (Pabd), detrusor pressure (Pdet) and urethral sphincter electromyography (EMG) activity, measured during a filling cytometry of a patient in a supine position;

FIG. 2 is a concept view for describing a urodynamic measurement apparatus according to an embodiment of the present disclosure;

FIG. 3 is a perspective view of a phantom for urodynamic measurement according to an embodiment of the present disclosure;

FIG. 4 is a perspective view of the phantom for urodynamic measurement of FIG. 3, with an upper surface cover removed from a case of the phantom;

FIG. 5 is a view for describing the upper surface cover of FIG. 4;

FIG. 6 is a view for describing a pressure provider installed on the upper surface cover of FIG. 4;

FIG. 7 is a view illustrating a graph that is shown on a display of the urodynamic measurement apparatus, when a first projection part of the upper surface cover of FIG. 4 is pressed;

FIG. 8 is a view illustrating a graph that is shown on the display of the urodynamic measurement apparatus, when a second projection part of the upper surface cover of FIG. 4 is pressed;

FIGS. 9 and 10 are views for describing a lower urinary tract part and a rectal part of the phantom for urodynamic measurement of FIG. 3;

FIG. 11 is a view for describing a volume adjusting part of the phantom for urodynamic measurement of FIG. 3;

FIGS. 12A and 12B are views for describing a compliance adjusting part of the phantom for urodynamic measurement of FIG. 3;

FIG. 13 is a view illustrating a graph that is shown on the display of the urodynamic measurement apparatus, when an inner space of a mesh pocket of the compliance adjusting part of FIG. 12 is made small and fluid is injected into a urinary bladder bag;

FIG. 14 is a view for describing a compliance adjusting part according to another embodiment of the present disclosure;

FIGS. 15 and 16 are views for describing a lower support part of the phantom for urodynamic measurement of FIG. 3;

FIGS. 17 and 18 are views for describing operations of the lower support part of FIG. 15;

FIG. 19 is a view for describing a posture adjusting part of the phantom for urodynamic measurement of FIG. 3;

FIG. 20 is a block diagram of the phantom for urodynamic measurement of FIG. 3;

FIG. 21 is a graph showing the intravesical pressure (Pves), abdominal pressure (Pabd), detrusor pressure (Pdet) and urethral sphincter electromyography (EMG) activity of a patient whose urine leaks when coughing;

FIG. 22 is a view for describing a fluid circulation part of the phantom for urodynamic measurement according to another embodiment of the present disclosure;

FIG. 23 is a perspective view of the phantom for urodynamic measurement according to another embodiment of the present disclosure;

FIG. 24 is a perspective view of the phantom for urodynamic measurement of FIG. 23, with the upper cover, lower urinary tract part, and rectal part removed;

FIG. 25 is a view of the upper cover of the phantom for urodynamic measurement of FIG. 23, seen from a lower side;

FIG. 26 is a view for describing operations of the lower support part of the phantom for urodynamic measurement of FIG. 23; and

FIG. 27 is a view where a urodynamic measurement apparatus is installed on the phantom for urodynamic measurement of FIG. 3.

### DETAILED DESCRIPTION

Hereinbelow, some embodiments of the present disclosure will be described in detail through the exemplary drawings. In adding reference numerals to components of each drawing, it should be noted that even if the components are displayed on different drawings, like reference numerals are used for like components as much as possible.

Further, in describing the embodiments of the present disclosure, if it is determined that a specific description of a related well-known configuration or a function interrupts the understanding of the embodiments of the present disclosure, detailed description thereof will be omitted.

Further, in describing the components of the present disclosure, terms such as a first, a second, A, B, (a), (b) and the like may be used. Such terms are merely used to distinguish those components from other components, and such terms do not limit the nature, sequence or order of the corresponding components.

Hereinbelow, a phantom for urodynamic measurement according to an embodiment of the present disclosure will be described in detail with reference to the accompanying drawings.

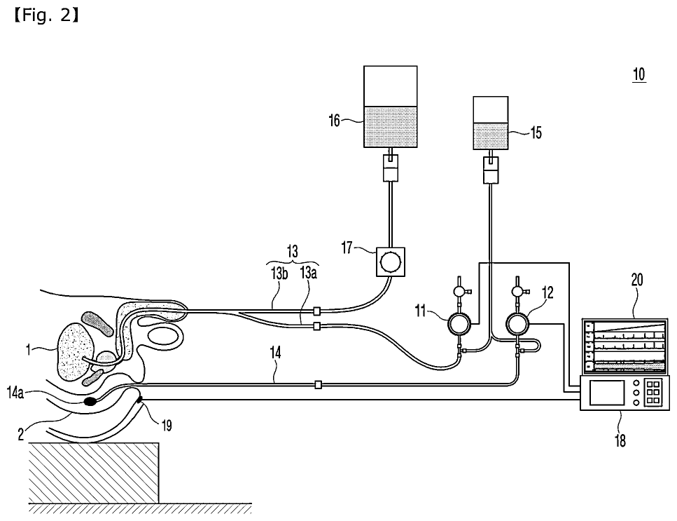

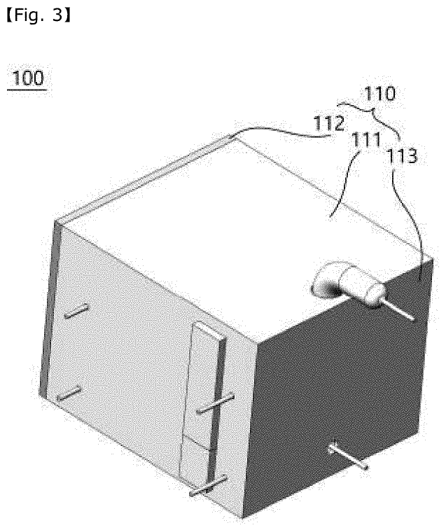

FIG. 2 is a concept view for describing a urodynamic measurement apparatus, and FIG. 3 is a perspective view of a phantom for urodynamic measurement according to an embodiment of the present disclosure.

Referring to FIGS. 2 and 3, a phantom for urodynamic measurement 100 of the present disclosure may perform various functions related to the functions of storing and emptying urine, that are necessary for urodynamic measurement, and may transmit various stimuli to a urodynamic measurement apparatus 10 to be used in tests for measuring various detailed indicators of urodynamic measurement.

In addition, the phantom for urodynamic measurement 100 may be used for educating medical staff who perform urodynamic measurement using the urodynamics measurement apparatus 10, and may be used in performance tests of newly developed urodynamic measurement apparatuses 10, etc.

Hereinbelow, the urodynamic measurement apparatus 10 will be described, and then the phantom for urodynamic measurement 100 of the present disclosure will be described in detail.

The urodynamic measurement apparatus 10 may include a first pressure transducer 11, a second pressure transducer 12, a urethral catheter 13, a rectal catheter 14, a first bag 15, a second bag 16, a pump 17, and a controller 18.

For urodynamic measurement, the urethral catheter 13 is inserted inside a patient's urinary bladder 1, and one end of the urethral catheter 13 is positioned inside the patient's urinary bladder 1.

The urethral catheter 13 may include a first catheter 13a that may be connected to the first pressure transducer 11, and a second catheter 13b that may be connected to the second bag 16.

The first catheter 13a of the urethral catheter 13 and the second catheter 13b of the urethral catheter 13 may each be independently formed inside one tube. That is, the urethral catheter 13 may have a form where two paths are provided in one tube.

Fluid such as physiological saline to be provided to the patient's urinary bladder 1 is filled inside the second bag 16. In addition, the second bag 16 is disposed in a higher place than the patient's urinary bladder 1.

The second bag 16 may be connected to the second catheter 13b, to supply the fluid such as physiological saline to the second catheter 13b, and the fluid such as physiological saline supplied to the second catheter 13b may flow into the patient's urinary bladder 1 and fill the patient's urinary bladder 1.

The fluid flowing from the second bag 16 to the patient's urinary bladder 1 may flow by gravity, or the pump 17 installed between the second bag 16 and the second catheter 13b may pump the fluid of the second bag 16 to the patient's urinary bladder 1 through the second catheter 13b.

The first pressure transducer 11 may be connected with the urethral catheter 13, and measure an internal pressure of the patient's urinary bladder 1.

The first pressure transducer 11 may measure the internal pressure of the patient's urinary bladder 1 during a filling phase in which the fluid such as physiological saline is supplied to the patient's urinary bladder through the second catheter 13b, and during a voiding phase in which the fluid such as physiological saline filled in the patient's urinary bladder is discharged through the urethra.

Fluid such as physiological saline is filled inside the first bag 15, and the first bag 15 is disposed in a higher place than the patient's urinary bladder 1.

When the fluid of the first bag 15 is injected into the first pressure transducer 11 and the first catheter 13a, from one end of the first catheter 13a, the fluid is discharged to the patient's urinary bladder, and at this point, the first pressure transducer 11 may measure the internal pressure of the patient's urinary bladder in the method of measuring the hydraulic pressure of the first catheter 13a.

For urodynamic measurement, the rectal catheter 14 is inserted inside the patient's rectum 2, and one end of the rectal catheter 14 may be positioned inside the patient's rectum 2.

At a tip portion of the rectal catheter 14, a rectal balloon 14a is provided, and the rectal balloon 14a may be filled with fluid such as physiological saline.

When the fluid of the first bag 15 is injected into the second pressure transducer 12 and the rectal catheter 14, the fluid flows into the rectal balloon 14a positioned at the tip of the rectal catheter 14. At this point, the second pressure transducer 12 may measure the abdominal pressure of the patient in the method of measuring the hydraulic pressure of the rectal catheter 14.

The urodynamic measurement apparatus 10 may further include an electromyography electrode 19.

The electromyography electrode 19 is attached near the patient's anus, and measures the activity of the urethral sphincter, and the measured electromyography activity may be used to determine whether a detrusor contraction is synergic or dyssynergic with the urethral sphincter activity.

The controller 18 may be connected with the first pressure transducer 11, the second pressure transducer 12, and the electromyography electrode 19, and based on signals received from the first pressure transducer 11, the second pressure transducer 12 and the electromyography electrode 19, the controller 18 may display the intravesical pressure (Pves) 1, abdominal pressure (Pabd) 2, detrusor pressure (Pdet) 3, and the urethral sphincter activity 14 through a display 20.

Hereinbelow, the phantom for urodynamic measurement according to an embodiment of the present disclosure will be described in detail with reference to the accompanying drawings.

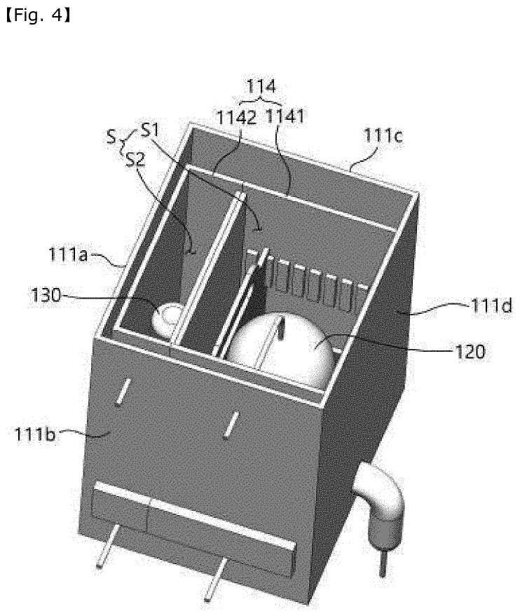

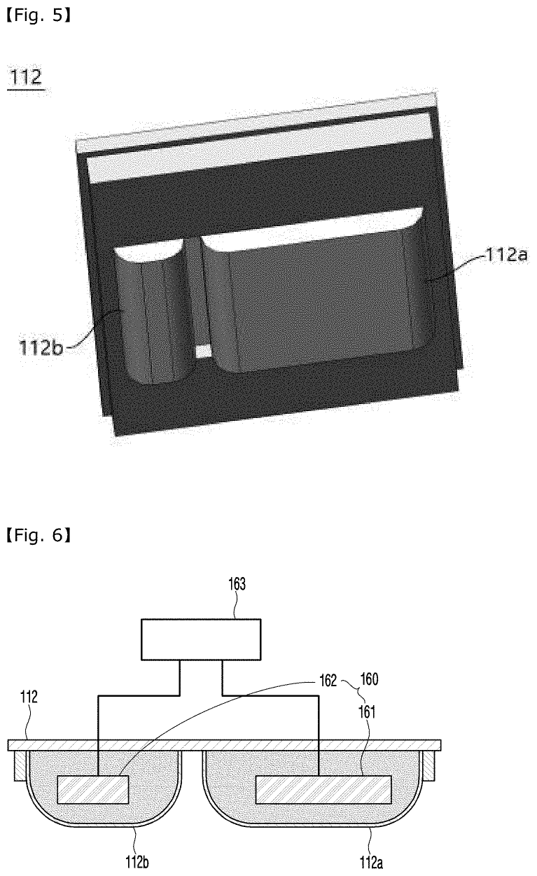

FIG. 4 is a perspective view of the phantom for urodynamic measurement of FIG. 3, with the upper surface cover removed from the case of the phantom for urodynamic measurement, and FIG. 5 is a view for describing the upper surface cover of FIG. 3.

Referring to FIGS. 4 and 5 further, the phantom for urodynamic measurement 100 of the present disclosure may include a pressure transmission case 110, a lower urinary tract system 120 (hereinafter referred to as “lower urinary tract part”) and a rectal part 130.

Inside the pressure transmission case 110, a sealed space S is formed so that the lower urinary tract part 120 and the rectal part 130 may be positioned therein.

In the space 5, the lower urinary tract part 120 is positioned in front of the rectal part 130 (right side in FIG. 4).

The space S is filled with fluid such as physiological saline, and the lower urinary tract part 120 and the rectal part 130 may be accommodated in the space S.

The pressure transmission case 110 may be elastically deformed in the direction from outside to the space S by external force. When the case 110 is being elastically deformed, the pressure of the fluid of the space S increases, and the increased pressure of the fluid of the space S is transmitted to the lower urinary tract part 120 and the rectal part 130.

The pressure transmission case 110 includes a container 111, 113 and an upper surface cover 112.

The container 111, 113 forms the space S that is upwardly open.

The upper surface cover 112 is installed at an upper side of the container 111, 113, and may shield the space S.

As illustrated, the pressure transmission case 110 includes the container 111, 113, and the upper surface cover 112, and the pressure transmission case 110 may form a cube or a cuboid, but the shape is not limited thereto, and thus, the pressure transmission case 110 may be formed as a single body sphere or polyhedron.

The container 111, 113 may include a lower surface cover 113, and a side surface cover 111 that is installed at an outer circumference of the lower surface cover 113, to form the space.

The upper surface cover 112 may shield an upper side of the side surface cover 111, and the lower surface cover 113 may shield a lower side of the side surface cover 111, to form the space S.

The side surface cover 111 may include a first cover 111a, a second cover 111b, a third cover 111c and a fourth cover 111d.

The first cover 111a forms a rear surface of the pressure transmission case 110, and the second cover 111b and the third cover 111c are connected to a left side end portion and to a right side end portion of the first cover 111a, respectively, and each end portion of the fourth cover 111d is connected to the second cover 111b and the third cover 111c.

The side surface cover 111 may be made of a material having elasticity.

The modulus elasticity of the side surface cover 111 may increase as it gets farther away from the portion adjacent to the lower urinary tract part 120.

That is, it is desirable that the modulus elasticity of the side surface cover 111 increases from the front towards the rear side.

Specifically, the modulus elasticity of the first cover 111a may be greater than that of the second cover 111b and the third cover 111c, and the modulus elasticity of the second cover 111b and the third cover 111c may be greater than the modulus elasticity of the fourth cover 111d.

The first cover 111a to the fourth cover 111d may be made of different materials, or may be made of the same material but have different thicknesses, to adjust the modulus elasticity.

The patient's urinary bladder 1 and the rectum 2 are positioned in the hypogastrium or the suprapubic region, and at a rear side of the urinary bladder 1 and the rectum 2, the pelvis is positioned.

Therefore, when the patient coughs, generally the patient's hypogastrium swells up to the front side.

The pressure transmission case 110 of the present disclosure may also have different modulus elasticity depending on the position of the side surface cover 111, and when pressure is applied from the upper surface cover 112 to the space S direction, the front side portion of the pressure transmission case 110 may be greatly deformed, and swell up to outside.

The space S may be divided into a first space S1, that is a lower urinary tract region where the lower urinary tract part 120 is positioned, and a second space S2, that is a rectal region where the rectal part 130 is positioned.

Specifically, an auxiliary side surface cover 114 installed in the space S may divide the space S into the first space S1 and the second space S2.

Moreover, the auxiliary side surface cover 114 may divide the space S physically into the first space S1 and the second space S2.

The auxiliary side surface cover 114 may be positioned inside the side surface cover 111, and the height of the auxiliary side surface cover 114 may be adjusted such that the first space S1 and the second space S2 are blocked from each other, or such that the first space S1 and the second space S2 communicate with each other.

Specifically, if an upper end of the auxiliary side surface cover 114 touches a lower surface of the upper surface cover 112, the first space S1 and the second space S may be blocked from each other, and when the upper end of the auxiliary side surface cover 114 is spaced apart from the lower surface of the upper surface cover 112, the first space S1 and the second space S2 may communicate with each other.

The auxiliary side surface cover 114 may include a first auxiliary side surface cover 1141 and a second auxiliary side surface cover 1142.

The first auxiliary side surface cover 1141 may be disposed on a circumference of the lower urinary tract part 120, and the upper side of the first auxiliary side surface cover 1141 may be shielded by a portion of the upper surface cover 112, and the lower portion of the first auxiliary side surface cover 1141 may be shielded by a portion of the lower surface cover 113, thereby forming the first space S1.

The second auxiliary side surface cover 1142 may be disposed on a circumference of the rectal part 130, and the upper side of the second auxiliary side surface cover 1142 may be shielded by a portion of the upper surface cover 112, and the lower portion of the second auxiliary side surface cover 1142 may be shielded by a portion of the lower surface cover 113, thereby forming the second space S2.

On the first auxiliary side surface cover 1141 and on the second auxiliary side surface cover 1142, an opening may be formed respectively, so that the first space S1 and the second space S2 may communicate with each other.

The first space S1 is positioned at a front side of the space S, and the second space S2 is positioned at a rear side of the space S.

In the first space S1, the lower urinary tract part 120 may be positioned, and in the second space S2, the rectal part 130 may be positioned.

When the upper surface cover 112 is pressed from outside of the upper surface cover 112 to the space S direction, the pressure of the fluid of the first space S1 and the pressure of the fluid of the second space S2 may increase.

The increased pressure of the fluid of the first space S1 may be transmitted to the lower urinary tract part 120, and the increased pressure of the fluid of the second space S2 may be transmitted to the rectal part 130.

In order for the pressure applied to the upper surface cover 112 to be transmitted from the space S to the lower urinary tract part 120 and the rectal part 130, the space S may be filled with fluid such as water and physiological saline, etc.

Therefore, when the upper surface cover 112 is pressed by external force in the space S direction, the pressure of the liquid of the space S increases.

Such increased pressure of the liquid of the space S may press the lower urinary tract part 120 and the rectal part 130, thereby increasing the internal pressure of the urinary bladder bag 121 of the lower urinary tract part 120 and the internal pressure of the rectal bag 131 of the rectal part 130.

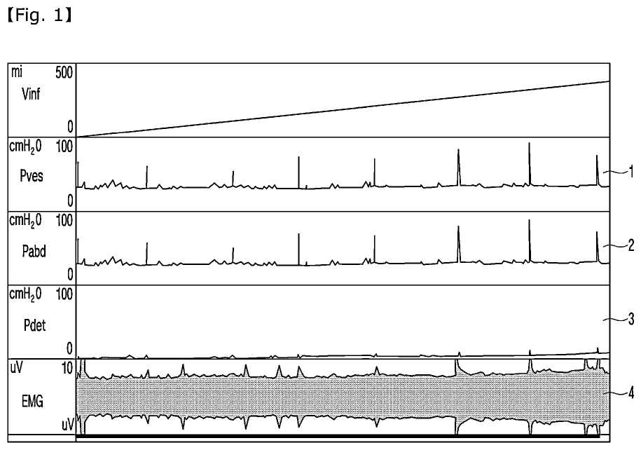

Referring to FIG. 1 again, when the patient coughs as per instructions from the medical staff performing the urodynamic measurement, pressure is transmitted to the patient's urinary bladder 11 and the rectum 2 at the same time, and the first pressure transducer 11 and the second pressure transducer 12 of the urodynamic measurement apparatus 10 will measure the patient's intravesical pressure (Pves) 1 and abdominal pressure (Pabd) 2, and display the result on the display 20.

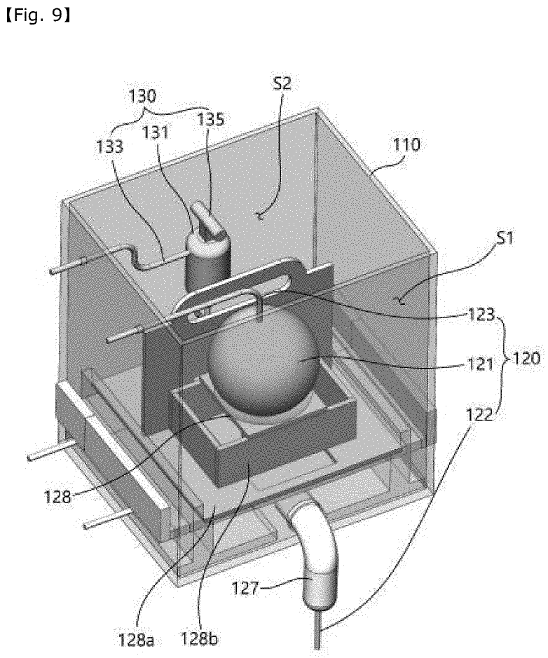

As such, when the upper surface cover 112 is pressed by external force in the space S direction, the first pressure transducer 11 and the second pressure transducer 12 of the urodynamic measurement apparatus 10 may display the pressure of the urinary bladder bag 121 and the rectal bag 131 on the display 20 as the patient's intravesical pressure (Pves) 1 and the abdominal pressure (Pabd) 2. In this case, the tip of the urethral catheter 13 is positioned inside the urinary bladder bag 121, and the end portion of the rectal catheter 14 is positioned inside the rectal bag 131 (see FIG. 9).

On a lower surface of the upper surface cover 112, a first projection part 112a that is projected to the first space S1, and a second projection part 112b that is projected to the second space S2 may be formed, respectively.

The surface of the first projection part 112a may be positioned adjacent to the urinary bladder of the lower urinary tract part 120, and the surface of the second projection part 112b may be positioned adjacent to the rectal part 130.

When the upper end of the auxiliary side surface cover 114 touches the lower surface of the upper surface cover 112, the first space S1 and the second space S2 are blocked from each other, and when the outer surface of the upper surface cover 112 at the first projection part 112a side is pressed, the first projection part 112a may increase the pressure of the fluid of the first space S1 and transmit the pressure to the lower urinary tract part 120, and when the outer surface of the upper surface cover 112 at the second projection part 112b side is pressed, the second projection part 112b may increase the pressure of the fluid of the second space S2, and transmit the pressure to the rectal part 130.

When the upper end of the auxiliary side surface cover 114 is spaced apart from the lower surface of the upper surface cover 112, and the first space S1 and the second space S2 communicate with each other, the pressure of the fluid of the space S1 and second space S2 become equivalent to each other.

Therefore, when the outer surface of the upper surface cover 112 at the first projection part 112a side is pressed, the liquid of the first space S1 will first press the lower urinary tract part 120, but the liquid pressure of the entirety of the space S will equally increase, and transmit the pressure to the lower urinary tract part 120 and the rectal part 130.

Conversely, when the outer surface of the upper surface cover 112 at the second projection part 112b side is pressed, the liquid that flew from the second projection part 112b will first press the rectal part 130, but thereafter, the pressure of the liquid of the entirety of the space S will equally increase, and transmit the pressure to the lower urinary tract part 120 and the rectal part 130.

FIG. 6 is a view for describing a pressure supplier installed on the upper surface cover of FIG. 4.

Referring to FIG. 6 further, the phantom for urodynamic measurement of the present disclosure may further include the pressure supplier 160.

The pressure supplier 160 may be installed inside each of the first projection part 112a and the second projection part 112b, and make the first projection part 112a and the second projection part 112b to swell up to the first space S1 and the second space S2, respectively.

For this purpose, the pressure supplier 160 may include a first air pocket 161, a second air pocket 162 and a pneumatic provider 163.

The first air pocket 161 may be installed inside the first projection part 112a, and the second air pocket 162 may be installed inside the second projection part 112b.

The pneumatic provider 163 may be connected to each of the first air pocket 161 and the second air pocket 162, and provide air to one or more of the first air pocket 161 and the second air pocket 162.

When the pneumatic provider 163 provides air to the first air pocket 161, the first air pocket 161 expands, and the first projection part 112a may transmit pressure to the fluid of the first space S1.

When the pneumatic provider 163 provides air to the second air pocket 162, the second air pocket 162 expands, and the second projection part 112b may transmit pressure to the fluid of the second space S2.

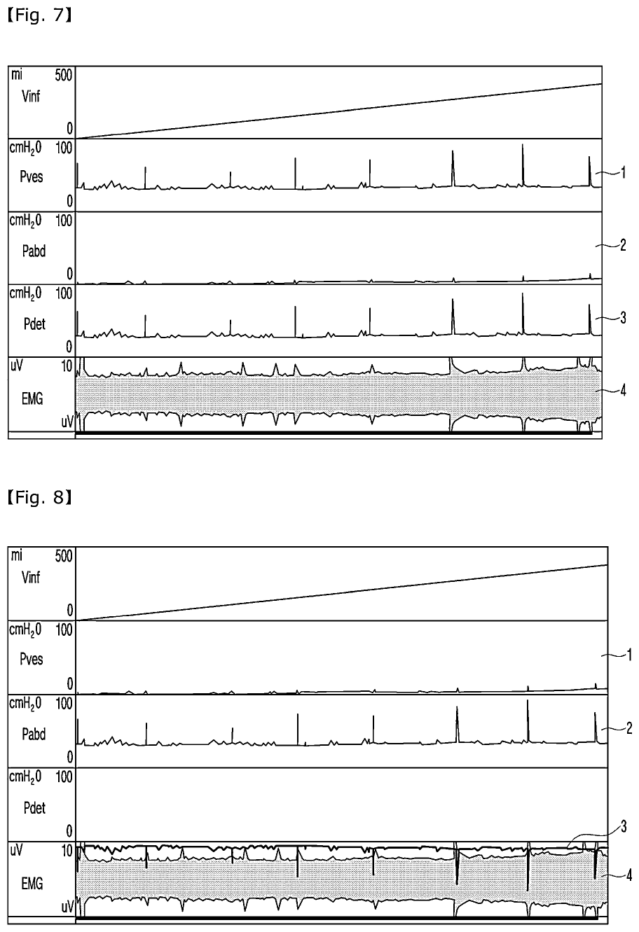

FIG. 7 is a view illustrating a graph that is shown on the display of the urodynamic measurement apparatus, when the first projection part side of the upper surface cover of FIG. 4 is pressed.

When the first projection part 112a side is repeatedly pressed for a certain period of time, a pressure that is higher than the pressure transmitted to the rectal bag 131 is repeatedly transmitted to the urinary bladder bag 121, and the pressure of the urinary bladder bag 121 measured in the first pressure transducer 11 becomes higher than the pressure of the rectal bag 131 measured in the second pressure transducer 12. As can be seen from FIG. 7, on the display 20 of the urodynamic measurement apparatus 10, the intravesical pressure (Pves) 1 shows a higher peak than the abdominal pressure (Pabd) 2, and the detrusor pressure 13 also has a high peak.

FIG. 8 is a view illustrating a graph that is shown on the display of the urodynamic measurement apparatus, when the second projection part side of the upper surface cover of FIG. 4 is pressed.

When the second projection part 112b side is repeatedly pressed for a certain period of time, a pressure that is higher than the pressure transmitted to the urinary bladder bag 121 is repeatedly transmitted to the rectal bag 131, and the pressure of the rectal bag 131 measured in the second pressure transducer 12 becomes higher than the pressure of the urinary bladder bag 121 measured in the first pressure transducer 11. As can be seen from FIG. 8, on the display 20 of the urodynamic measurement apparatus 10, the abdominal pressure (Pabd) 2 shows a higher peak than the intravesical pressure (Pves) 1. The detrusor pressure 13 is displayed to have a negative peak.

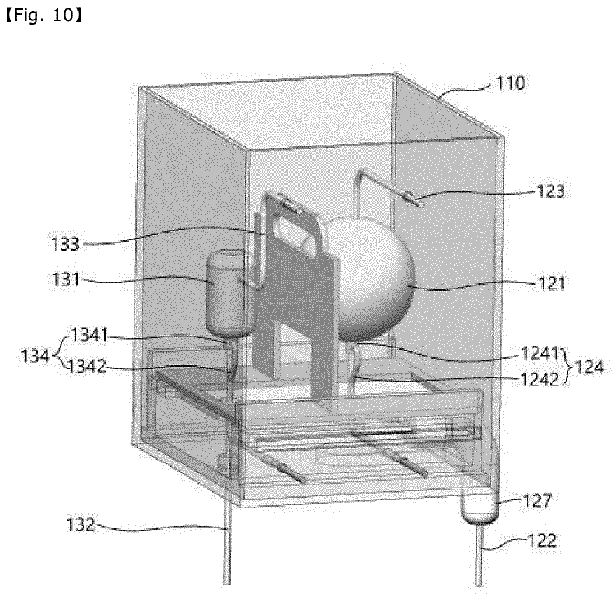

FIGS. 9 and 10 are views for describing the lower urinary tract part and the rectal part of the phantom for urodynamic measurement of FIG. 3.

Referring to FIGS. 9 and 10 further, the lower urinary tract part 120 may include the urinary bladder bag 121, a urethral hose 122, a first air discharge pipe 123, and a first sphincter part 124.

The urinary bladder bag 121 may be disposed in the first space S1, and may be made of a material that is expandable and contractable when fluid flows in or flows out.

On a lower portion of the first space S1, a plate 128 having curved surface that is concaved downwardly may be installed, and the urinary bladder bag 121 may be disposed in the first space S1 in such a way that the urinary bladder bag 121 is seated on the plate 128.

Therefore, the urinary bladder bag 121 may be elastically deformable, and may have any shape that can accommodate fluid therein.

The urethral hose 122 is connected to the other end of the urinary bladder bag 121.

Specifically, the urethral hose 122 may be connected to a lower end of the urinary bladder bag 121.

Inside the urethral hose 122, a first path is formed.

The first path may be blocked or opened.

The first path may be blocked or opened. Therefore, the inside of the urinary bladder bag 121 may be connected to outside through the first path, or blocked from outside.

For example, the urethral hose 122 may be made of a flexible material such as a rubber tube.

Through the first path of the urethral hose 122, fluid may be supplied to the inside of the urinary bladder bag 121, or the fluid inside the urinary bladder bag 121 may be discharged to the outside.

In addition, through the first path, the urethral catheter 13 may be inserted into the urinary bladder bag 121.

One end of the urethral hose 122 may communicate with a lower end of the urinary bladder bag 121, and the other end may be exposed to outside of the pressure transmission case 110.

In a lower portion of the pressure transmission case 110, a penis tube 127 is provided.

Inside the penis tube 127, a tunnel may be formed, and the other end of the urethral hose 122 may be disposed in an inner tunnel of the penis tube 127, and the other end of the urethral hose 122 may be exposed to outside.

For urodynamic measurement education or performance test of the urodynamic measurement apparatus 10 of the present disclosure, the urethral catheter 13 may be inserted into the other end of the urethral hose 122 and moved to the inside of the urinary bladder bag 121 so that the tip of the urethral catheter 13 is positioned inside the urinary bladder bag 121. The urethral catheter 13 may measure the pressure of the fluid inside the urinary bladder bag 121.

The first sphincter part 124 is installed to surround the urethral hose 122 at one end of the urethral hose 122 at a lower side of the urinary bladder bag 121.

The first sphincter part 124 may adjust the size of the first path of the urethral hose 122 by adjusting the degree of pressing one end of the urethral hose 122 from the outside to the inside.

The first sphincter part 124 may include a first sphincter tube 1241 and a first air supplier 1242.

The urethral hose 122 may be disposed inside the first sphincter tube 1241, and the first sphincter tube 1241 may adjust the size of the first path by applying pressure from the circumference to the inside of the urethral hose 122.

The first air supplier 1242 may supply air to the first sphincter tube 1241. When the air is supplied to the first sphincter tube 1241, the first sphincter tube 1241 may swell up, and the outer circumference of the urethral hose 122 that is inside the first sphincter tube 1241 may be pressed to the inside.

When the degree by which the first sphincter tube 1241 swells up is adjusted according to the amount of air supplied from the first air supplier 1242 to the first sphincter tube 1241, the size of the first path may be adjusted, and the amount of the fluid being discharged from the urinary bladder bag 121 and the flow rate of the fluid being discharged from the urinary bladder bag 121 may be adjusted.

The first air discharge pipe 123 is connected to one end of the urinary bladder bag 121.

Specifically, the first air discharge pipe 123 is connected to an upper end of the urinary bladder bag 121.

The first air discharge pipe 123 may be connected to the upper end of the urinary bladder bag 121, and discharge the gas collected in the inner upper side of the urinary bladder bag 121 to the outside.

Therefore, the first air discharge pipe 123 may discharge only the gas from the inside of the urinary bladder bag 121 to the outside, so that the inside of the urinary bladder bag 121 is filled with only liquid.

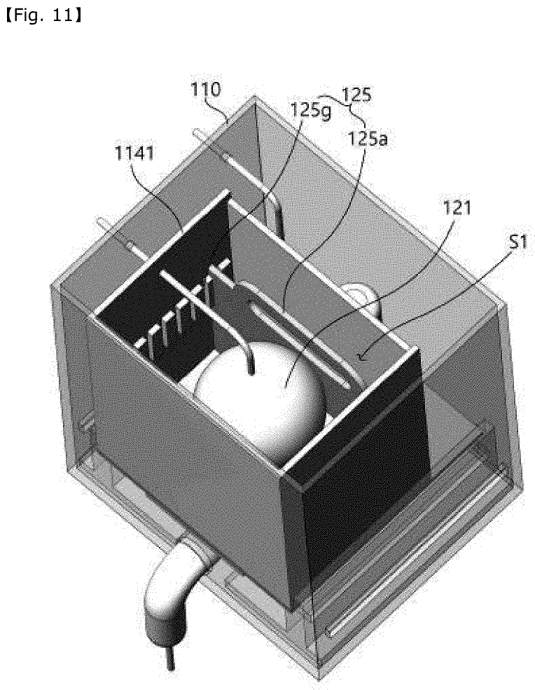

FIG. 11 is a view for describing a volume adjusting part of the phantom for urodynamic measurement of FIG. 3.

Referring to FIG. 11 further, the lower urinary tract part 120 may further include the volume adjusting part 125.

The volume adjusting part 125 may adjust the amount of the fluid being injected into the urinary bladder pocket 121 by adjusting the size of the space S in which the urinary bladder bag 121 is accommodated.

Specifically, the volume adjusting part 125 may adjust the size of the first space S1 where the urinary bladder bag 121 is positioned, and the urinary bladder bag 12 may be elastically deformed within only the first space S1 of which the size has been adjusted, and thus the amount of fluid being injected into the urinary bladder bag 12 may also be adjusted.

The amount of the fluid being injected into the urinary bladder bag 121 may be adjusted by adjusting the size of the first space S1.

The volume adjusting part 125 may include a plurality of insertion grooves 125g provided in an inner surface of the first auxiliary side surface cover 1141, and an adjusting plate 125a inserted into one or more of the plurality of insertion grooves 125g, to adjust the size of the first space S1.

In the present disclosure, it has been described that the adjusting plate 125a is inserted into one or more of the plurality of insertion grooves 125g and fixed to a set portion of the first space S1. However, it can be any configuration such as a magnet, an electromagnet, a screw, a pin, and an adhesive material, that may be selectively fixed to the set portion inside the first space S1.

When fluid flows into the urinary bladder bag 121 and the urinary bladder bag 121 is elastically deformed in the first space S1, the adjusting plate 125a may touch the urinary bladder bag 121 and adjust the deformed shape of the urinary bladder bag 121.

If the adjusting plate 125a is positioned and fixed such that the elastically deformable range of the urinary bladder bag 121 is reduced in the first space S1, for example, when the adjusting plate 125a is moved close to the urinary bladder bag 121 in a horizontal direction on the drawings and fixed thereto, when the fluid flows into the urinary bladder bag 121 and the urinary bladder bag 121 expands, the urinary bladder bag 121 touches the adjusting plate 125a in the horizontal direction, and the deformation degree in an up and down direction in the drawings, that is a portion in the urinary bladder bag 121 that does not touch the adjusting plate 125a (hereinafter referred to as “deformed portion”) becomes larger. Since the deformed portion is controlled to deform only within the elasticity limit, the amount of the fluid flowing into the urinary bladder bag 121 decreases.

To summarize, if the adjusting plate 125a touches the urinary bladder bag 121, when fluid is flowing into the urinary bladder bag 121, the deformable portion in the urinary bladder bag 121 decreases, and the deformed portion is controlled to be deformed only within the elasticity limit, and thus the amount of fluid flowing into the urinary bladder bag 121 decreases.

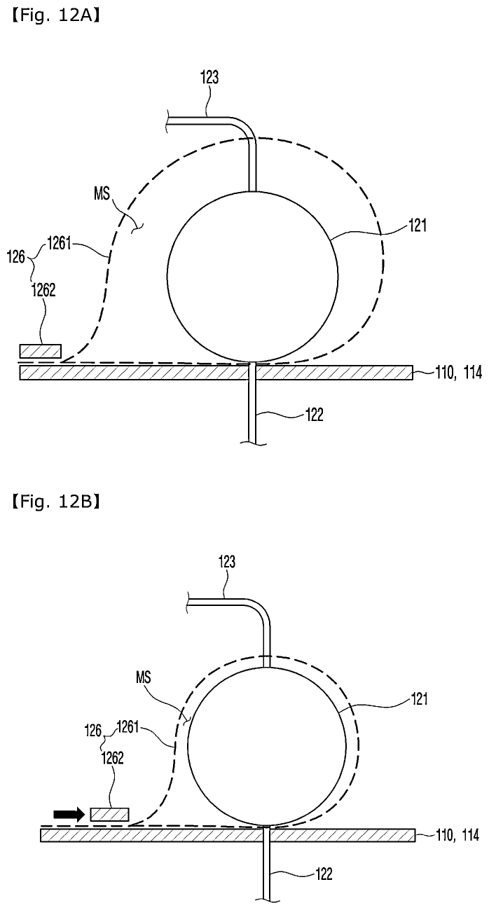

FIGS. 12A and 12B are views for describing a compliance adjusting part of the phantom for urodynamic measurement of FIG. 3.

Referring to FIGS. 12A and 12B further, the lower urinary tract part 120 may further include a compliance adjusting apparatus 126 (hereinafter referred to as “compliance adjusting part”).

The compliance adjusting part 126 may include a pocket 1261 and a fixing pin 1262.

The pocket 1261 may be a bag made of a net that can accommodate the urinary bladder bag 121 therein.

The present disclosure may adjust the elasticity modulus of the urinary bladder bag 121 by adjusting the size of the inner space S of the pocket 1261.

Specifically, the pocket 1261 is fixed to the inner surface of the pressure transmission case 110 or the auxiliary side surface cover 114, forming the first space S1, and the fixing pin 1262 may adjust the degree by which the pocket 1261 is fixed to the inner surface of the pressure transmission case 110 or the auxiliary side surface cover 114 inside the first space S1, thereby adjusting the size of the inner space MS of the pocket 1261.

If the inner space MS of the pocket 1261 becomes small, when fluid is injected into the urinary bladder pocket 121, the outer surface of the urinary bladder bag 121 touches the pocket 1261, and thereafter, the elasticity modulus of the urinary bladder bag 121 increases significantly, and the pressure of the fluid inside the urinary bladder bag 121 increases.

That is, by the compliance adjusting part 126, the pressure of the fluid inside the urinary bladder bag 121 may be adjusted, and the urethral catheter 13 and the first pressure transducer 11 may measure the pressure of the fluid inside the urinary bladder bag 121.

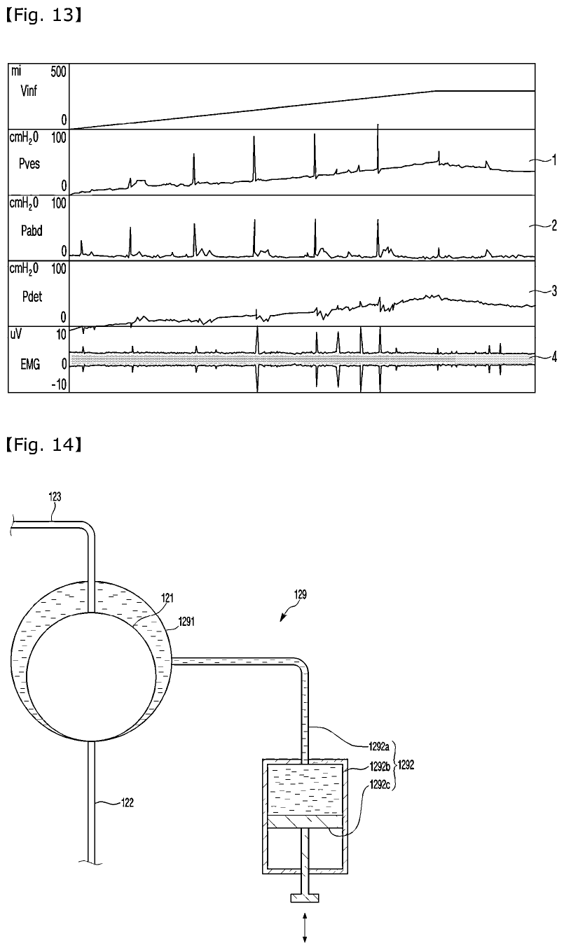

FIG. 13 is a view illustrating a graph that is shown on the display of the urodynamic measurement apparatus when the inner space of a mesh pocket of the compliance adjusting part of FIG. 12 is reduced and fluid is injected into the urinary bladder bag.

When the fixing pin 1262 fixes the pocket 1261 to the inner surface of the first space S1 so that the size of the inner space MS of the pocket 1261 gets smaller and then fluid flows into the urinary bladder bag 121, the outer surface of the urinary bladder bag 121 touches the pocket 1261, and then the elasticity modulus of the urinary bladder bag 121 increases significantly.

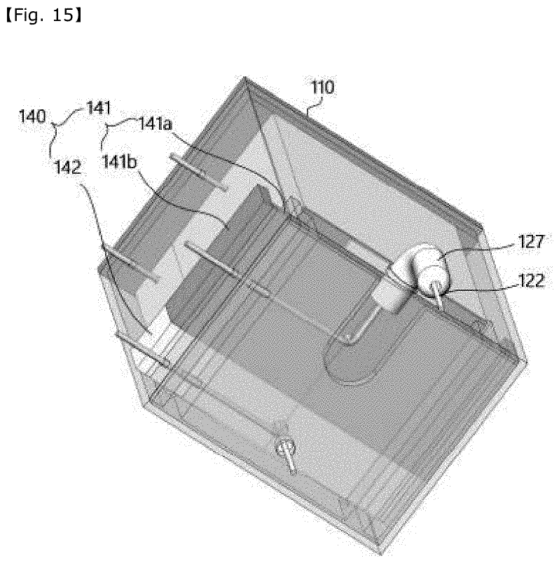

Here, there is no change to the pressure of the rectal part 130 measured in the second pressure transducer 12, but the pressure of the lower urinary tract part 120 measured in the first pressure transducer 11 increases, and thus as can be seen from FIG. 14, on the display 20 of the urodynamic measurement apparatus 10, the intravesical pressure (Pves) 1 and the detrusor pressure (Pdet) 3 are displayed to increase.

FIG. 14 is a view for describing the compliance adjusting part according to another embodiment of the present disclosure.

Referring to FIG. 14 further, the lower urinary tract part 120 may further include a compliance adjusting part 129.

The compliance adjusting part 129 may include a fluid bag 1291 and a fluid adjusting part 1292.

The fluid bag 1291 may accommodate the urinary bladder bag 121 therein.

The fluid bag 1291 may be made of a material having a greater elasticity modulus than that of the urinary bladder bag 121. Therefore, when fluid is supplied to the urinary bladder bag 121 and the urinary bladder bag expands, the outer surface of the urinary bladder bag 121 may touch the inner surface of the fluid bag 1291, and the pressure of the fluid inside the urinary bladder bag 121 may increase significantly.

Meanwhile, fluid may be filled between the inner surface of the fluid bag 1291 and the outer surface of the urinary bladder bag 121, thereby adjusting the degree by which the urinary bladder bag 121 expands.

To describe specifically, when a large amount of fluid is injected between the inner surface of the fluid bag 1291 and the outer surface of the urinary bladder bag 121, even when a small amount of fluid is injected into the urinary bladder bag 121, the fluid between the inner surface of the fluid bag 1291 and the outer surface of the urinary bladder bag 121 will apply pressure to the urinary bladder bag 121. Accordingly, the pressure of the fluid inside the urinary bladder bag 121 may increase significantly.

Conversely, when a small amount of fluid is injected between the inner surface of the fluid bag 1291 and the outer surface of the urinary bladder bag 121, the fluid between the inner surface of the fluid bag 1291 and the outer surface of the urinary bladder bag 121 may apply pressure to the urinary bladder bag 121 when a large amount of fluid is supplied in the urinary bladder bag 121.

That is, depending on the amount of fluid being injected between the inner surface of the fluid bag 1291 and the outer surface of the urinary bladder bag 121, the pressure of the fluid inside the bag 121 may differ.

The fluid adjusting part 1292 may adjust the amount of fluid being injected between the inner surface of the fluid bag 1291 and the outer surface of the urinary bladder bag 121.

Specifically, the fluid adjusting part 1292 may be connected with the fluid bag 1291, to supply fluid to the inside of the fluid bag 1291, or discharge the fluid inside the fluid bag 1291 to outside.

The fluid adjusting part 1292 may include a connecting hose 1292a, a cylinder 1292b, and a piston 1292c.

The connecting hose 1292a connects the cylinder 1292b and the fluid bag 1291.

Inside of the cylinder 1292b may be filled with fluid. The fluid inside the cylinder 1292b may move to the fluid bag 1292 through the connecting hose 1292a, or the fluid of the fluid bag 1291 may move to the cylinder 1292b through the connecting hose 1292a.

The piston 1292c may be installed inside the cylinder 1292b such that it can move reciprocally.

When the piston 1292c moves to one side (lower side on the drawing) such that the pressure inside the cylinder 1292b decreases, the fluid of the fluid bag 1291 flows into the cylinder 1292b through the connecting hose 1292a, and when the piston 1292c moves to the other side (upper side on the drawing) such that the pressure inside the cylinder 1292b decreases, the fluid inside the cylinder 1292b may move to the fluid bag 1291 through the connecting hose 1292a.

Referring to FIGS. 9 and 10 again, the rectal part 130 may include a rectal bag 131, a guide hose 132, a second air discharge pipe 133, and a second sphincter part 134.

The rectal bag 131 may be disposed in the second space S2, and may be made of a material that is elastically deformable, such as balloon.

The rectal bag 131 may be disposed in the second space S2 in such a manner that a fixing stick 135 attached to an upper surface of the rectal bag 131 is fixed to the second auxiliary side surface cover 1142.

Specifically, the fixing stick 135 may be formed in a T shape, with a lower end of the fixing stick 135 attached to the rectal bag 131, and both ends of the fixing stick 135 fixed to the second auxiliary side surface cover 1142.

Inside of the rectal bag 131 may be in a state that is filled with fluid such as water, physiological saline and the like.

The guide hose 132 is connected to the other end of the rectal bag 131.

Specifically, one end of the guide hose 132 is connected with the lower end of the rectal bag 131, and the other end is exposed to outside.

The second air discharge pipe 133 is connected to one end of the rectal bag 131.

Specifically, the second air discharge pipe 133 is connected to the upper end of the rectal bag 131.

Through the second air discharge pipe 133, the air inside the rectal bag 131 may be discharged to outside.

For education of the urodynamic measurement or performance test of the urodynamic measurement apparatus 10 of the present disclosure, the rectal catheter 14 may be inserted into the other end of the guide hose 132 and move up until the inside of the rectal bag 131 such that the tip of the rectal catheter 14 is positioned inside the rectal bag 131.

The tip portion of the rectal catheter 14 may be provided with a rectal balloon 14a, and the rectal balloon 14a may be filled with fluid such as physiological saline and the like.

The second sphincter part 134 is installed to surround the guide hose 132 at one end portion of the guide hose 132 at a lower side portion of the urinary bladder bag 121.

The second sphincter part 134 may adjust the size of the second path of the guide hose 132 by adjusting the degree by which the end portion of the guide hose 132 is pressed from outside to inside.

The second sphincter part 134 may include a second sphincter tube 1341 and a second air supplier 1342.

The guide hose 132 may be disposed inside the second sphincter tube 1341, and the second sphincter tube 1341 may adjust the size of the second path by applying pressure from the circumference of the guide hose 132 to inside.

The second air supplier 1342 may supply air to the second sphincter tube 1341. When the air is supplied to the second sphincter tube 1341, the second sphincter tube 1341 swells up, and the circumference of the guide hose 132 that is inside the second sphincter tube 1341 may be pressed to inside.

When the degree by which the second sphincter tube 1341 swells up is adjusted according to the amount of air supplied from the second air supplier 1342 to the second sphincter tube 1341, the size of the second path is adjusted as well, thereby adjusting the degree of leakage of liquid such as water and physiological saline in the inner space S of the rectal bag 131.

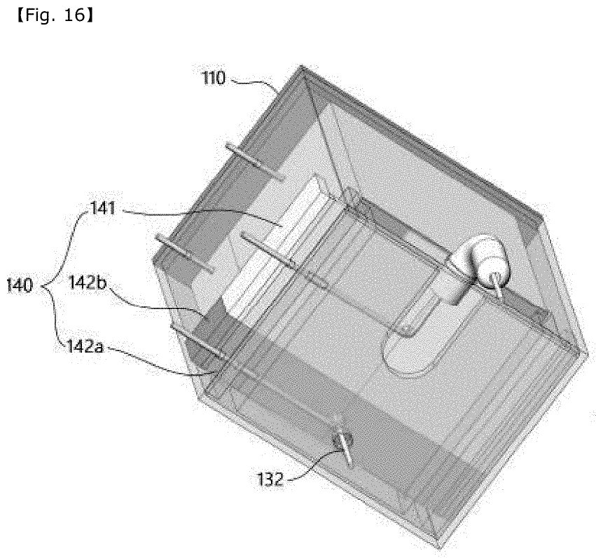

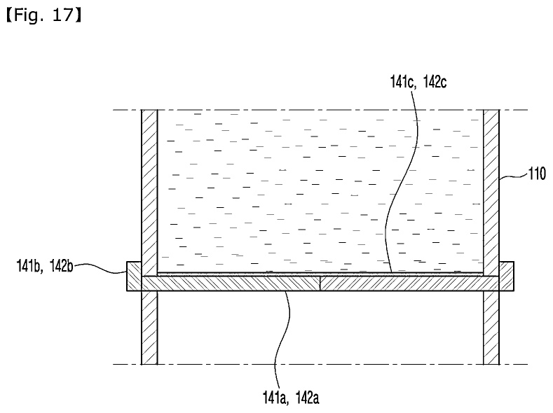

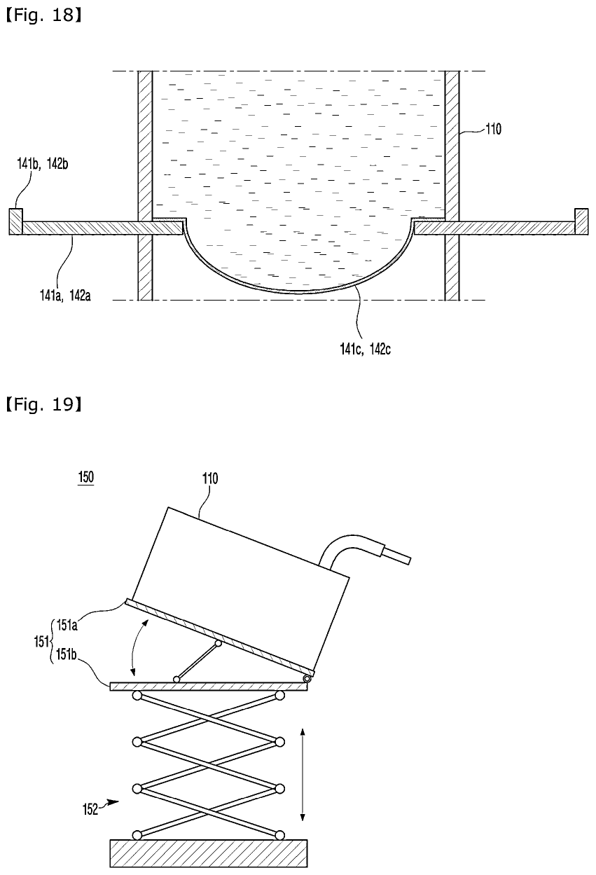

FIGS. 15 and 16 are views for describing a lower support part of the phantom for urodynamic measurement of FIG. 3, and FIGS. 17 and 18 are views for describing operations of the lower support part.

Referring to FIGS. 15 to 18 further, the phantom for urodynamic measurement 100 may further include the lower support part 140.

The lower support part 140 may include sliding plates 141a, 142a, and pressure absorption members 141c, 142c.

The sliding plates 141a, 142a are installed such that they are slidable on a lower portion of the space S. The sliding plates 141a, 142a may be positioned on the lower portion of the space S, or may slide to outside of the case 110.

When the upper surface cover 112 is pressed in the space S direction, the fluid of the space S transmits the pressure transmitted from the upper surface cover 112 to the lower support part 140.

Here, when the sliding plates 141a, 142a are positioned on the lower portion of the space S, the pressure absorption members 141c, 142c are not deformed, and the pressure of the fluid of the space S increases.

However, when the sliding plates 141a, 142a slide to outside of the case 110, the pressure absorption members 141c, 142c absorb the pressure of the fluid of the space S, thereby preventing the pressure of the fluid of the space S from increasing.

Specifically, when the upper surface cover 112 is pressed from outer side of the upper surface cover 112 to the space S direction, by the fluid of the space S, the pressure absorption members 141c, 142c extend downwardly. In such a case, since the pressure of the fluid of the space S does not increase, the lower urinary tract part 120 and the rectal part 130 are prevented from being pressed by the fluid of the space S.

Further, in the present disclosure, according to the degree by which the sliding plates 141a, 142a slide to outside of the case 110, when the upper surface cover 112 is pressed in the space S direction, the degree of increase of the pressure of the fluid in the space S may be adjusted.

When the degree of increase of the pressure of the fluid in the space S is adjusted as aforementioned, the degree by which the lower urinary tract part 120 and the rectal part 130 are pressed by the fluid of the space S are adjusted as well.

The lower support part 140 may include a first lower support part 141 and a second lower support part 142.

The first pressure absorption member 141c of the first lower support part 141 may be disposed at a lower side of the first space S1, and absorb the increased pressure of the liquid of the first space S1, and the first absorption member 141c of the second lower support part 142 may be disposed on a lower side of the second space S2, and absorb the increased pressure of the liquid of the second space S2.

The first lower support part 141 may include a first sliding plate 141a, a first knob 141b, and the first pressure absorption member 141c.

The first sliding plate 141a is installed such that it is slidable on the lower portion of the first space S1, and the first knob 141b is connected to one side of the first sliding plate 141a, and thus when a user pulls the first knob 141b away from the pressure transmission case 110, or pushes the first knob 141b towards the pressure transmission case 110 direction, the first sliding plate 141a slides on the lower portion of the first space S1.

The first pressure absorption member 141c may be disposed on an upper side of the first sliding plate 141a, and block the lower portion of the first space S1.

The first pressure absorption member 141c may be made of a material having a smaller elasticity modulus than that of the pressure transmission case 110.

When the first sliding plate 141a slides to outside of the pressure transmission case 110, the first pressure absorption member 141c is extended to the lower portion of the first space S1 by the liquid of the first space S1, and even when the upper surface cover 112 is pressed from outside of the upper surface cover 112 to the space S direction, the increased pressure of the liquid of the first space S1 may be absorbed to the first pressure absorption member 141c at the lower portion of the first space S1 without being transmitted to the lower urinary tract part 120.

The second lower support part 142 may include a second sliding plate 142a, a second knob 142b, and a second pressure absorption member 142c.

The second sliding plate 142a is installed such that it is slidable on a lower portion of the second space S2, and the second knob 142b is connected to one side of the second sliding plate 142a, and thus when the user pulls the second knob 142b away from the pressure transmission case 110, or pushes the second knob 142b towards the pressure transmission case 110 direction, the second sliding plate 142a slides on the lower portion of the second space S2.

The second pressure absorption member 142c may be disposed on an upper side of the second sliding plate 142a, and may block a lower portion of the second space S2.

The second pressure absorption member 142c may be made of a material having a smaller elasticity modulus than that of the pressure transmission case 110.

When the second sliding plate 142a slides to the outside of the pressure transmission case 110, the second pressure absorption member 142c is extended to the lower portion of the second space S2 by the liquid of the second space S2, and even when the upper cover 112 is pressed from outside of the upper cover 112 to the space S direction, the increased pressure of the liquid of the second space S1 may be absorbed to the second pressure absorption member 142c at a lower portion of the second space S2 without being transmitted to the rectal part 130.

FIG. 19 is a view for describing a posture adjusting part of the phantom for urodynamic measurement.

Referring to FIG. 19 further, the phantom for urodynamic measurement 100 may further include the posture adjusting part 150.

For urodynamic measurement education or performance test of the urodynamic measurement apparatus 10 of the present disclosure, the posture adjusting part 150 may rotate the pressure transmission case 110, or transfer the pressure transmission case 110 to an up/down direction.

The pressure transmission case 110 may be mounted onto the rotation part 151, and the rotation part 151 may move the pressure transmission case 110 such that a supine position, sitting position, standing position and the like are copied just as the patient's posture of measurement.

The posture adjusting part 150 may include the rotation part 151 and an elevation part 152.

The rotation part 151 may include a bracket 151a and an angle adjusting part 151b.

The pressure transmission case 110 is mounted onto the bracket 151a. The angle adjusting part 151b is mounted onto the elevation part 152.

The angle adjusting part 151b may adjust the inclination of the bracket 151a.

The bracket 151a may be hinge-coupled to the angle adjusting part 151b, and the angle adjusting part 151b may rotate the bracket 151a around the hinge.

The elevation part 152 may elevate the rotation part 151. The elevation part 152 may be any configuration capable of elevating the rotation part 151 such as a hydraulic cylinder 1292b, gear, and robot, etc.

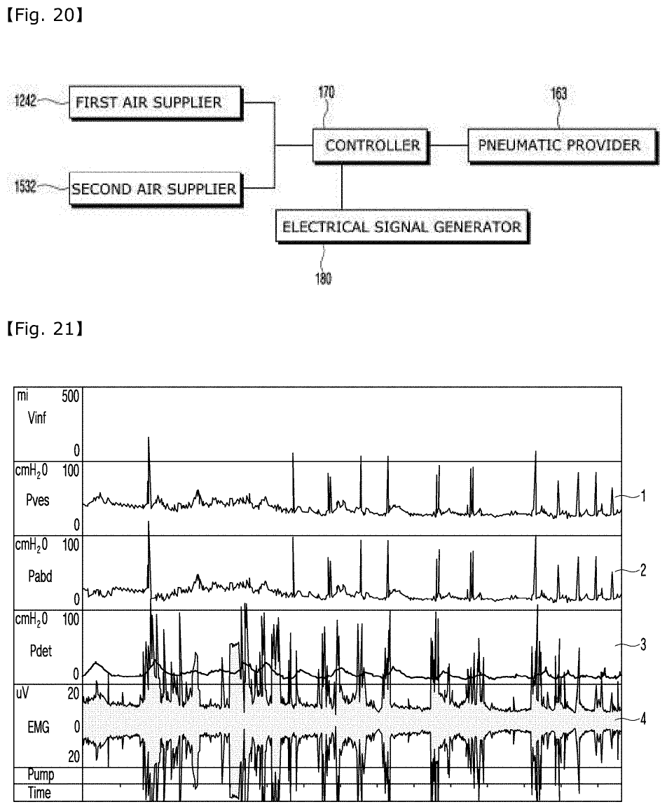

FIG. 20 is a block diagram of the phantom for urodynamic measurement of FIG. 3, and FIG. 21 is a graph showing the intravesical pressure (Pves), abdominal pressure (Pabd), detrusor pressure (Pdet) and urethral sphincter electromyography (EMG) activity of a patient whose urine leaks when coughing.

Referring to FIGS. 19 and 20 further, the phantom for urodynamic measurement 100 may further include an electrical signal generator 180 and a controller 170.

For urodynamic measurement education or performance test of the urodynamic measurement apparatus 10 of the present disclosure, the electromyography electrode 19 may be attached to the electrical signal generator 180, and the electrical signal generator 180 may generate an electrical signal to be measured in the electromyography electrode 19.

The controller 170 may be connected to each of the first air supplier 1242, the first air supplier 1242, the pneumatic provider 163, and the electrical signal generator 180, and control the first air supplier 1242, the first air supplier 1242, the pneumatic provider 163, and the electrical signal generator 180, respectively.

For example, with the supplying of air by the second air supplier 1342 to the second sphincter tube 1342 being maintained, the controller 170 may control to reduce the amount of air that the first air supplier 1242 supplies to the first sphincter tube 1241, increase the air pressure that the pneumatic provider 163 provides to the first air pocket 161, and reduce the air pressure that the pneumatic provider 163 provides to the second air pocket 162. At the same time, the controller 170 may control so that an electrical signal is generated from the electrical signal generator 180.

When the controller 170 controls the first air supplier 1242, the first air supplier 1242, the pneumatic provider 163 and the electrical signal generator 180 as aforementioned, as shown in FIG. 20, the fluid inside the urinary bladder bag 121 may be discharged to outside through the urethral hose 122, and on the display 20 of the urodynamic measurement apparatus 10, a graph shows where the intravesical pressure (Pves) 1 and abdominal pressure (Pabd) 2 repeatedly show peaks, and the detrusor pressure (Pdet) 3 increases. In addition, the urethral sphincter activity graph also shows change.

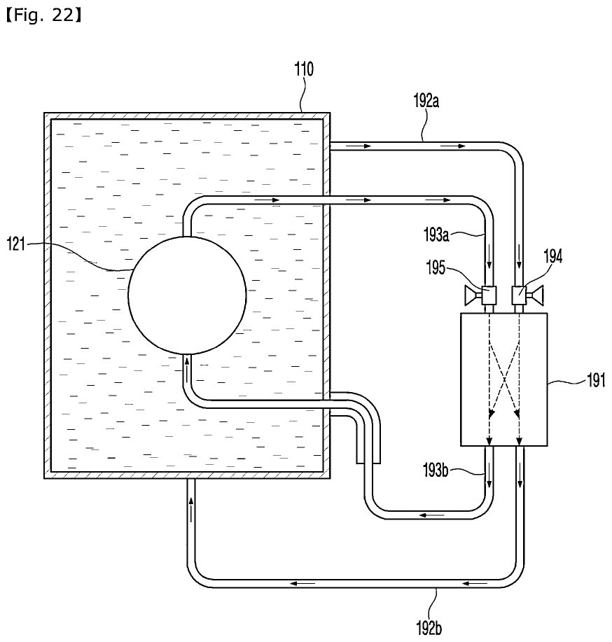

FIG. 22 is a view for describing a fluid circulator of the phantom for urodynamic measurement according to another embodiment of the present disclosure.

Referring to FIG. 22, the phantom for urodynamic measurement 100 according to another embodiment of the present disclosure may further include a fluid circulation system 100 (hereinafter referred to as “fluid circulator”).

The fluid circulator 190 is connected to the pressure transmission case 110 and the urinary bladder bag 121 of the lower urinary tract part, and circulates fluid of the urinary bladder bag 121 and the pressure transmission case 110, to control the amount of the fluid being injected into the urinary bladder bag 121 and the pressure transmission case 110.

Specifically, the fluid circulator 190 may control such that the sum of the amount L1 of the injected liquid of the urinary bladder bag 121 and the amount L2 of the liquid injected into the pressure transmission case 10 is equivalent to a set reference value L. Since the reference value L is constant, when the amount L1 of the injected liquid of the urinary bladder bag 121 decreases, the amount L2 of the liquid injected into the pressure transmission case 110 increases, and when the amount L1 of the injected liquid of the urinary bladder bag 121 increases, the amount L2 of the liquid injected into the pressure transmission case 110 decreases.

The fluid circulator 190 may include a circulation pump 191, a first discharge path 192a, a first injection path 192b, a second discharge path 193a, a second injection path 193b, a first adjusting valve 194, and a second adjusting valve 195.

The circulation pump 191 may circulate fluid.

The first discharge path 192a may be connected with the pressure transmission case 110 and the circulation pump 191 at each side, and the liquid of the pressure transmission case 110 may move to the circulation pump 191 through the first discharge path 192a.

The first injection path 192b may be connected to the circulation pump 191 and the pressure transmission case 110 at each side, and the liquid of the circulation pump 191 may move to the pressure transmission case 110 through the first injection path 192b.

The second discharge path 193a may be connected to the urinary bladder bag 121 and the circulation pump 191 at each side, and the liquid of the urinary bladder bag 121 may move to the circulation pump 191 through the second discharge path 193a.

The second injection path 193b may be connected to the circulation pump 191 and the urinary bladder bag 121 at each side, and the liquid of the circulation pump 191 may move to the urinary bladder bag 121 through the second injection path 193b.

The first adjusting valve 194 may be installed in the first discharge path 192a, and may block the first discharge path 192a, or adjust the flow rate of the liquid moving from the first discharge path 192a.

The second adjusting valve 195 may be installed in the second discharge path 193a, and may block the second discharge path 193a, or adjust the flow rate of the liquid moving from the second discharge path 193a.

With the circulation pump 191 operating, when the first adjusting valve 194 is adjusted to block the first discharge path 192a, or the flow rate of the liquid moving from the first discharge path 192a is reduced, the amount L2 of the liquid injected into the pressure transmission case 110 increases, and the amount L1 of the injected liquid of the urinary bladder bag 121 decreases.

In addition, with the circulation pump 191 operating, when the second adjusting valve 195 is adjusted to block the second discharge path 193a or the flow rate of the liquid moving from the second discharge path 193a is reduced, the injected amount L1 of the liquid of the urinary bladder bag 121 decreases, and the amount L2 of the liquid injected into the pressure transmission case 110 increases.

With the sum of the amount L1 of the injected liquid of the urinary bladder bag 121 and the amount L2 of the liquid injected into the pressure transmission case 110 controlled to be equivalent to the set reference value L, the fluid circulator 190 may adjust the degree of opening and closing of the first adjusting valve 194 and the second adjusting valve 195, to control the volume of the urinary bladder bag 121 and the compliance degree of the urinary bladder bag 121.

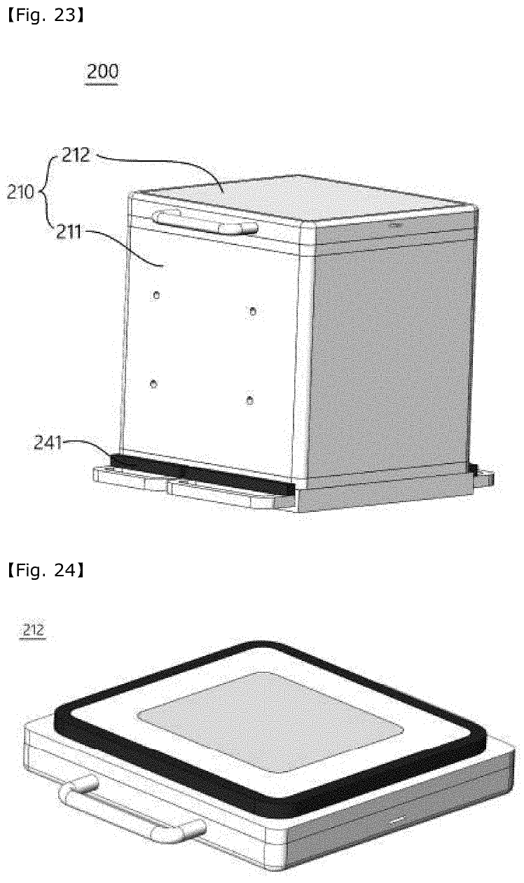

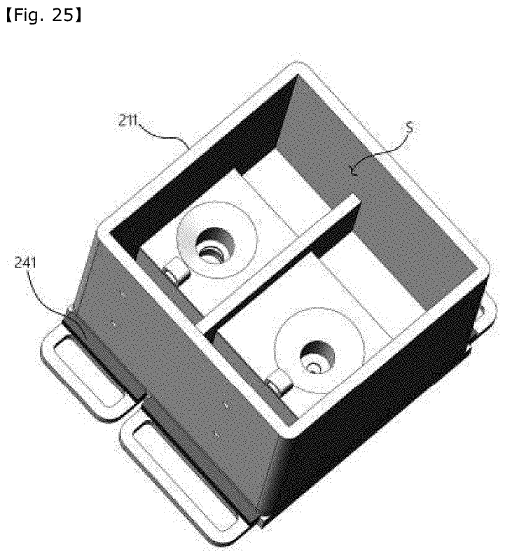

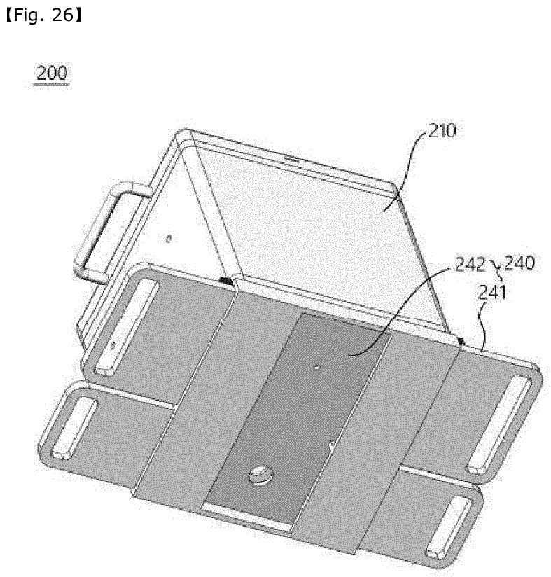

FIG. 23 is a perspective view of the phantom for urodynamic measurement according to another embodiment of the present disclosure, FIG. 24 is a perspective view of the phantom for urodynamic measurement of FIG. 23, with the upper cover, the lower urinary tract part and the rectal part removed, FIG. 25 is a view of the upper cover of the phantom for urodynamic measurement of FIG. 23, seen from the lower side, and FIG. 26 is a view for describing operations of the lower support part of the phantom for urodynamic measurement of FIG. 23.

Referring to FIGS. 23 to 25, inside a pressure transmission case 210 of a phantom for urodynamic measurement 200 of the present disclosure, a sealed space S is formed, in which the lower urinary tract part 120 and the rectal part 130 may be positioned.

The space S may be filled with fluid such as physiological saline, and the lower urinary tract part 120 and the rectal part 130 may be accommodated in the space S.

The pressure transmission case 210 may be elastically deformed from outside to the space S direction by external force, and when the case 210 is elastically deformed, the pressure of the fluid of the space S increases, and the increased pressure of the fluid of the space S is transmitted to the lower urinary tract part 120 and the rectal part 130.

Specifically, the pressure transmission case 310 includes a container 211 and an upper surface cover 212. The upper surface cover 212 may be made of an elastic material such as silicon.

When the upper surface cover 112 is pressed from outside to the space S direction, the pressure of the fluid of the space S increases, and the increased pressure of the fluid of the space S may be transmitted to the lower urinary tract part 120 and the rectal part 130.

Referring to FIG. 26 further, a lower support part 240 of the phantom for urodynamic measurement 200 of the present disclosure may include a sliding plate 241 and a pressure absorption member 242.

The sliding plate 241 is installed in one pair, and such that it is slidable on the lower portion of the space S. The sliding plate 241 may be positioned on the lower portion of the space S, or may slide to outside of the case 110.

When the upper surface cover 112 is pressed from outside to the space S direction, the fluid of the space S transmits the pressure transmitted from the upper surface cover 112 to the lower support part 140.

Here, when the sliding plate 241 is positioned on the lower portion of the space S, the pressure absorption member 242 is not deformed, and the pressure of the fluid of the space S increases.

However, when the sliding plate 241 slides to the outside of the case 110, the pressure absorption member 242 absorbs the fluid pressure of the space S, to prevent the pressure of the fluid of the space S from increasing.

Therefore, according to the degree by which the sliding plate 241 slides to the outside of the case 210, when the upper surface cover 212 is pressed in the space S direction, the degree of increase of the pressure of the fluid of the space S may be adjusted.

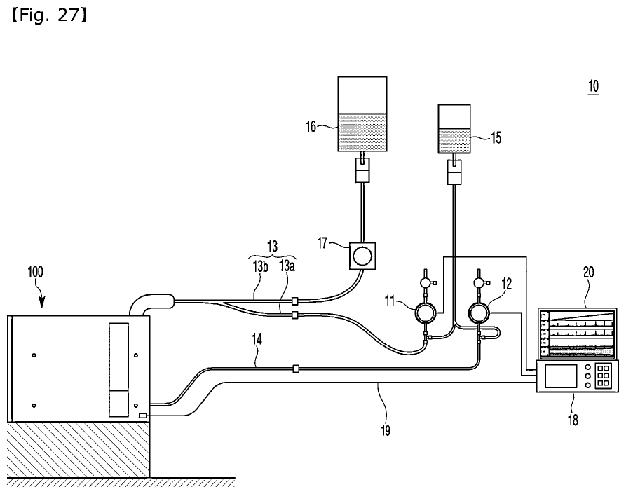

FIG. 27 is a view of a urodynamic measurement apparatus installed on the phantom for urodynamic measurement of FIG. 3.

Referring to FIG. 27, the urodynamic measurement apparatus 10 may be installed on the phantom for urodynamic measurement 100.

Specifically, the urethral catheter 13 may be inserted into the urinary bladder bag 121 of the lower urinary tract part 120, and the rectal catheter 14 may be inserted into the rectal bag 131 of the rectal part 130. In addition, the electromyography electrode 19 of the urodynamic measurement apparatus 10 may be attached to the electrical signal generator 180.

The controller 170 may control the internal pressure of the urinary bladder bag 121, the internal pressure of the rectal bag 131, the degree of pressing of the first sphincter part 124, the degree of pressing of the second sphincter part 134, and the electrical signal generated in the electrical signal generator 180. In addition, the urodynamic measurement apparatus 10 may measure the internal pressure of the urinary bladder bag 121, the internal pressure of the rectal bag 131, and the electrical signal from the electrical signal generator 180, being controlled in the controller 170, and display the same on the display 20.

That is, according to various scenarios of the lower urinary tract dysfunction, the controller 170 may control the internal pressure of the urinary bladder bag 121, the internal pressure of the rectal bag 131, the degree of pressing of the first sphincter part 124, the degree of pressing of the second sphincter part 134, and the electrical signal being generated in the electrical signal generator 180, and the urodynamic measurement apparatus 10 may measure and display the same through the display 20.

For example, when the controller 170 repeatedly expands the first projection part 112a side for a certain period of time so that the internal pressure of the urinary bladder bag 121 increases, as illustrated in FIG. 7, on the display 20 of the urodynamic measurement apparatus 10, the intravesical pressure (Pves) 1 will show a higher peak than the abdominal pressure (Pabd) 2, and the detrusor pressure (Pdet) 3 will also show a high peak.

In addition, when the controller 170 repeatedly expands the second projection part 112b side for a certain period of time so that the internal pressure of the rectal bag 131 increases, as can be seen from FIG. 8, on the display 20 of the urodynamic measurement apparatus 10, the abdominal pressure (Pabd) 2 shows a higher peak than the intravesical pressure (Pves) 1. The detrusor pressure 13 is shown to have a negative peak.

In addition, when the controller 170 controls the compliance adjusting part 129, to increase the fluid inside the fluid bag 1291, thereby increasing the pressure of the fluid of the urinary bladder bag 121, as shown in FIG. 14, on the display 20 of the urodynamic measurement apparatus 10, the intravesical pressure (Pves) 1 and the detrusor pressure (Pdet) 3 are displayed to increase.

In addition, when the controller 170 controls to increase the pressing force of the second sphincter part 134 to press the guide hose 132, and controls to decrease the pressing force of the first sphincter tube 1241 to open the first path of the urethral hose 122, and controls to repeatedly and greatly expand the first projection part 112a side for a certain period of time, and controls to repeatedly and slightly expand the second projection part 112b side for a certain period of time, and controls to generate an electrical signal from the electrical signal generator 180, the display 20 of the urodynamic measurement apparatus 10 shows a graph where the abdominal pressure (Pabd) 2 and the intravesical pressure (Pves) 1 repeatedly show peaks, and the detrusor pressure (Pdet) 3 increases. In addition, a change occurs in the graph of the urethral sphincter activity 14 as well.

In the above, even though it has been described that all components constituting an embodiment of the present disclosure operate in combination or combined into one, the present disclosure is not necessarily limited to this embodiment. That is, as long as it is within the range of the purpose of the present disclosure, one or more of all the components may be selectively combined and operate.

In addition, all terms including technical or scientific terms described above have the same meaning as commonly understood by those of ordinary skill in the art to which the present disclosure belongs, unless otherwise defined. Terms commonly used, such as those defined in the dictionary, should be interpreted as being consistent with the contextual meaning of the related art, and should not be interpreted in an ideal or excessively formal meaning unless explicitly defined in the present disclosure.

In addition, the above description is merely illustrative of the technical idea of the present disclosure, and those of ordinary skill in the art to which the present disclosure pertains may make various modifications and variations within a range that does not depart from the essential characteristics of the present disclosure. Therefore, the embodiments disclosed in the present disclosure are not intended to limit the technical spirit of the present disclosure, but to explain, and the scope of the technical spirit of the present disclosure is not limited by these embodiments. The protection scope of the present disclosure should be interpreted by the claims below, and all technical ideas within the scope equivalent thereto should be construed as being included in the scope of the present disclosure.

What is claimed is:

1. A phantom for urodynamic measurement that sends measurement stimulus to a urodynamic measurement apparatus, the phantom comprising:
a lower urinary tract part for providing pressure to a urethral catheter of the urodynamic measurement apparatus; and
a rectal part that is positioned adjacent to the lower urinary tract part, and that provides pressure to a rectal catheter of the urodynamic measurement apparatus.

2. The phantom for urodynamic measurement according to claim 1,
wherein the lower urinary tract part comprises a urinary bladder bag;
a first air discharge pipe connected to one end of the urinary bladder bag;
a urethral hose connected to the other end of the urinary bladder bag; and
a first sphincter part surrounding the urethral hose.

3. The phantom for urodynamic measurement according to claim 1,
further comprising a case for accommodating the lower urinary tract part and the rectal part,
wherein the case comprises an auxiliary side surface cover for physically separating the lower urinary tract part and the rectal part from each other.

4. The phantom for urodynamic measurement according to claim 1,
wherein the rectal part comprises a rectal bag;
a second air discharge pipe connected to one end of the rectal bag;
a guide hose connected to the other end of the rectal bag; and
a second sphincter part that surrounds the guide hose.

5. The phantom for urodynamic measurement according to claim 1, comprising an electrical signal generator for generating an electrical signal to an electromyography electrode of the urodynamic measurement apparatus.

6. The phantom for urodynamic measurement according to claim 1, further comprising a posture adjusting part for adjusting an angle of the case and a height of the case.
